(19) United States
(12) Plant Patent Application Publication
(10) Pub. No.: US 2016/0255756 P1
(43) Pub. Date: Sep. 1, 2016

(54) Apple Tree Named 'PreA129'

(50) ;

(71) Applicant: Allan G. White, Havelock North (NZ)

(72) Inventor: Allan G. White, Havelock North (NZ)

(21) Appl. No.: 14/998,811

(22) Filed: Feb. 18, 2016

Related U.S. Application Data
(60) Provisional application No. 62/176,507, filed on Feb. 19, 2015.

(51) Int. Cl. A01H 5/00

(52) U.S. Cl. PLT/161

(57) ABSTRACT

A new and distinct apple tree named ‘PremA129’ is disclosed. The new apple is notable for its attractive appearance, superb texture, improved flavor and long shelf life.

### STATEMENT REGARDING FEDERALLY SPONSORED RESEARCH OR DEVELOPMENT

None.

### LATIN NAME OF THE GENUS AND SPECIES OF THE PLANT CLAIMED

Malus domestica Mill.

### VARIETY DENOMINATION

‘PREMA129’

### BRIEF DESCRIPTION OF THE VARIETY

The new variety of apple tree ‘PremA129’ was created in the course of a planned breeding program carried out at The New Zealand Institute for Plant and Food Research, Hawkes Bay, New Zealand. ‘PremA129’ originated as a result of a controlled cross of ‘Scired’ (seed parent, unpatented) and ‘PremA280’ (pollen parent, U.S. Plant Pat. No. 19,762). ‘PremA129’ was selected as a single plant from among a population of seedlings derived from the parents. Asexual propagation by budding was first carried out in New Zealand. The variety has since been observed over a number of asexually propagated generations, and has been found to remain true to type.

‘PremA129’ is distinguished from its parent ‘Scired’ by the following characteristics:

| | Scired | PremA129 |
|---|---|---|
| Maturity | Late | Mid-season |
| Fruit Size | Medium | Large |
| Russet Sensitivity | Medium-High | Low |

### BRIEF DESCRIPTION OF THE PHOTOGRAPHS

FIG. 1 shows the fruit of a ‘PremA129’ apple tree; and

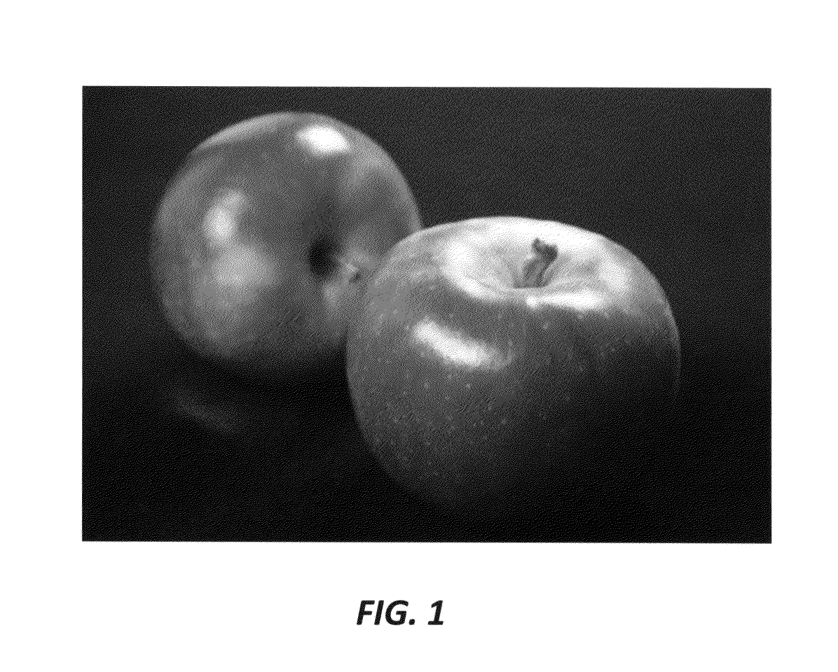

FIG. 2 shows a ‘PremA129’ apple tree.

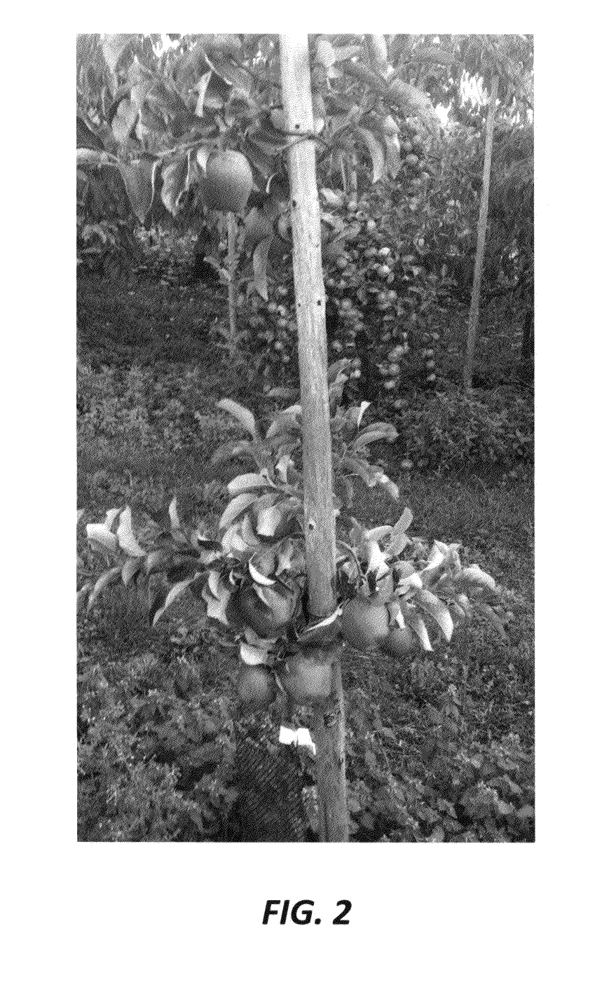

### DETAILED BOTANICAL DESCRIPTION OF THE VARIETY

The following detailed botanical description is based on observations made during the 2014 growing season at New Zealand. All colors are described according to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Type — Ramified; Vigor — Moderate; Habit — Semi-Spreading; Height — 200 cm; Diameter — 85 cm; Trunk diameter at 30 cm above the graft union) — 4 cm; Bark texture — Smooth; Bark coloration — 202D; Lenticels — Size 0.4 cm; Lenticel Color — 150D.
Branch: Length — 30 cm; Diameter — 1.5 cm; Crotch angle — 70°; Color — over color 202D; Base color — 174A; Lenticels — Size — 0.2 cm; Lenticel color — 150D; Quantity per inch²— 10.
1 Year old shoot: Length — 30 cm; Color — 174A; Thickness — 0.8 cm; Internode length — 4 cm; Lenticel size — 0.1 cm; Lenticel color — 150D; Quantity per inch²— 12.
Flower buds: Quantity per spur — 5; Length — 1.1 cm; Width — 0.7 cm; Color — 64C.
Flowers: Color at balloon stage — N66D; Diameter of open flower — 5 cm; Relative position of petal margin — Not touching; Depth — 0.5 cm; Number per cluster — 5.
Petals: Number per flower — 5; Length 2.5 cm; Width — 1.5 cm; Margin — Smooth; Color of upper surface — 155C with N66D and 68D edges; Color of lower surface — 155C with 68D edges.
Pistils: Size — 0.9 cm; Color — 155C.
Anthers: Quantity — 15; Size — 0.1 cm; Color of pollen — 2B.
Stigma: Size — 0.1 cm; Color — 2B.
Style: Size — 0.8 cm; Color — 155C.
Ovary: Size — Width — 0.4 cm, Length — 0.6 cm; Color — 138B.
Bloom: Date of first bloom — April 4^{th}; Date of full bloom — April 10^{th}.
Pedicel: Width — 0.1 cm; Length — 2.5 cm; Color — 194B.
Sepals: Quantity — 5; Color — 194B; Length 1 cm; Width — 0.5 cm.
Leaves: Length — 8 cm; Width — 4 cm; Blade margin — Serrated; Apex — Pointed; Base shape — round; Color of top surface — 144A; Color of bottom surface — 147C; Attitude in relation to shoot — 70°.
Petiole: Length — 4 cm; Diameter — 0.2 cm; Color — 148D.
Fruit: Shape — Conical; Ribbing — None; Depth of eye basin — 1 cm; Width of eye basin — 2 cm; Length of stalk — 2.5 cm; Thickness of stalk — 0.3 cm; Width of stalk cavity — 1.5 cm; Depth of stalk cavity — 0.8 cm; Size of lenticels — 0.1 cm; Background color of skin 150C; over color of skin — 46A and 34A; Relative area of over color — 95%;
Pattern of over color — solid; Flesh texture — fine; Juiciness — Moderate; Flesh color — 155A; Stem color — 145B
Seeds: Quantity per fruit — 12; Length 0.7 cm; Width — 0.4 cm; Color — 175B.
Use: Fresh Market.

We claim:

1. A new and distinct apple tree substantially as shown and described herein.
